(19) United States
(12) Patent Application Publication
(10) Pub. No.: US 2005/0260146 A1
(43) Pub. Date: Nov. 24, 2005

(54) Set of at least two solid compositions for application to the skin, to the lips, to the nails, and/or to hair

(76) Inventor: Xavier Blin, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/100,514

(22) Filed: Apr. 7, 2005

Related U.S. Application Data
(60) Provisional application No. 60/564,965, filed on Apr. 26, 2004.

(30) Foreign Application Priority Data
Apr. 8, 2004 (FR) 0450715

(51) Int. Cl. A61K 7/021; A61K 7/025

(52) U.S. Cl. 424/63; 424/64

(57) ABSTRACT

The present disclosure relates to a set of at least two solid makeup compositions for application to the skin, to the lips, to the nails, or to hair, the range comprising: at least one first composition having a first color due at least in part to the presence of at least one first composite pigment; and at least one second composition having a second color different from the first color, due at least in part to the presence of at least one second composite pigment different from the at least one first composite pigment, and wherein the at least one first and at least one second composite pigments comprise particles comprising a core, which is at least partially coated with at least one coloring substance; with the proviso that the coloring substances of the at least one first and at least one second composite pigments are different.

This non provisional application claims the benefit of French Application No. 04/50715 filed on Apr. 8, 2004, and U.S. Provisional Application No. 60/564,965 filed on Apr. 26, 2004, the contents of both of which are incorporated herein by reference.

The present disclosure relates to a set of at least two solid makeup cosmetic compositions for application to the skin, including the mucous membranes, for instance to the lips, to the nails, or to hair.

The present disclosure also relates for example, to a set of at least two solid compositions that are presented in stick form, e.g. lipsticks.

In order to make a set of compositions of different colors, it is known to incorporate pigments in such compositions in order to confer different colors thereto. The pigments may be of the organic or of the inorganic type and they may be of different kinds in at least two compositions within a given set. The different natures of the pigments incorporated in compositions of stick form can lead to variation in the mechanical properties of such sticks, for instance, because of the different ways in which the pigments behave with respect to other ingredients in the composition, for example oils.

Thus, there exists a need for a set of at least two solid compositions that present different colors but can have similar mechanical properties.

Accordingly, the present disclosure relates to a set of at least two solid makeup compositions for application to the skin, to the lips, to the nails, and/or to hair, comprising:
at least one first composition having a first color due at least in part to the presence of at least one first composite pigment; and
at least one second composition having a second color different from the first color, due at least in part to the presence of at least one second composite pigment different from the at least one first composite pigment; wherein the at least one first and at least one second composite pigments comprise particles comprising a core, which is at least partially coated with at least one coloring substance;
with the proviso that the coloring substances of the at least one first and at least one second composite pigments are different, and wherein the difference in hardness between the at least one first and at least one second compositions can be less than or equal to 25 grams (g) or 0.245 newtons (N), such as less than or equal to 20 g (0.196 N).

The cores of the at least one first composite pigment and at least one second composite pigments may be such that the difference in hardness between the at least one first and at least one second compositions is less than or equal to 25 grams (g) or 0.245 newtons (N), such as less than or equal to 20 g (0.196 N).

The present disclosure can make it possible to obtain a set of at least two compositions having different colors but of hardnesses that are relatively close, thus making it possible to build up a set of compositions that are uniform in hardness.

For example, the at least one first and at least one second composite pigments can comprise cores made of the same material.

A suitable tint for each composition in the set can be obtained in a variety of manners, for example by mixing the at least one first and at least one second composite pigments of the present disclosure, said pigments having different colors, and/or by the presence of a plurality of organic coloring substances in the coating of the cores of the at least one first and at least one second composite pigments, said organic coloring substances being mixed or being in the respective layers of the coating.

The set of at least two solid compositions of the present disclosure can further comprise at least one physiologically acceptable medium. As used herein, the term “physiologically acceptable medium” is understood to mean a non toxic medium that can be applied to the skin, to the lips, to the nails, or to hair of human beings. The physiologically acceptable medium will be adapted to the nature of the surface onto which each composition is to be applied, and to the form in which each composition is intended to be packaged.

As used herein, the term “cosmetic composition” is understood to mean a composition as defined in Council Directive 93/35/EEC dated 14 Jun. 1993.

The set of at least two solid compositions may comprise at least three compositions, or even at least five compositions, wherein each of the compositions have a different color, wherein the difference in hardness between each differently colored composition being less than or equal to 25 g.

As used herein, the term “difference in hardness between each differently colored composition” is understood to mean that the difference between the lowest hardness and the highest hardness of the compositions making up the set of at least two solid compositions is less than or equal to 25 g.

The cores of the at least first and at least second composite pigments can be inorganic. The at least one first and at least one second coloring substances coating the at least one first and at least one second composite pigments can be a coating of organic coloring material, thus making it possible to benefit from a relatively high coloring power.

The at least two solid compositions for the set may be, for instance, for application to the lips, and by way of example they may be in the form of sticks. Under such circumstances, the set of at least two solid compositions can be separately packaged, for example, in mechanisms comprising at least two portions capable of turning relative to each other in order to make the sticks move.

The set of at least two solid compositions can have ingredients that are the same apart from the composite pigments. For instance, the at least one first and at least one second compositions can comprise the same solvents and/or the same waxes.

In one aspect of the present disclosure, the at least one first and at least one second compositions can have no additional coloring substance other than the at least one first and at least one second composite pigments.

In one embodiment of the present disclosure, at least one first and/or at least one second composition can comprise at least one additional coloring substance other than the at least one first and at least one second composite pigments, wherein the at least one first and/or at least one second composite pigments are present in an amount greater than or equal to that of the additional coloring substance, for example.

In still another embodiment of the present disclosure, the at least one first and at least one second composite pigments are not interference pigments. As used herein, the term “interference pigment” is understood to mean, for example, a pigment comprising a superposition of layers of constant thickness of materials selected to produce optical interferences. An example of an interference pigment is disclosed in U.S. Pat. No. 6,428,773.

In one embodiment of the present disclosure, the at least one first and at least one second organic coloring substances are not melanin.

The saturation C* of the at least one first and at least one second composite pigments may be greater than or equal to 30, measured according to the following protocol.

Protocol for Measuring the Saturation C* of the Composite Pigment:

The color values a* and b* in the CIE L*a*b* colorspace of the at least one first and at least one second composite pigments are measured as follows.

The composite pigment in a raw state is compacted in a rectangular cup having dimensions of 2×1.5 cm and a depth of 3 mm, by applying a pressure of 100 bars.

The a* and b* values of the compacted pigment are measured with a Minolta 3700d spectrophotometer, in mode specular excluded under illuminant D65 and medium aperture. The saturation is computed as C*=(a*²+b*²)1/2.

Measuring Hardness

In order to determine the hardness of a solid composition, a stick of said composition is prepared having a circular section with a diameter of 12.7 millimeters (mm). The stick is cast 24 hours (h) before performing the measurements and it is conserved at a temperature of 20° C.

Hardness can be measured by the “cheesewire” method which comprises cutting the stick transversely using a rigid tungsten wire having a diameter of 250 micrometers (μm) and causing the wire to advance relative to the stick at a speed of 100 mm/min. The hardness corresponds to the maximum shear force exerted by the wire on the stick at 20° C., with this force being measured by means of a DFGS2 dyanometer sold by the supplier Indelco-Chatillon. The hardness is expressed in grams weight.

Composite Pigments

Structure

The at least one first and at least one second composite pigments of the present disclosure can be made up of particles comprising a core, which is at least partially coated with at least one coloring substance.

At least one binder can optionally be used, for example, to contribute to fixing the coloring substance on the core.

The cores of the at least one first and/or at least one second composite pigments can be inorganic.

The at least one first and/or at least one second coloring substances of the at least one first and/or at least one second composite pigments can be organic.

The at least one first and/or at least one second composite pigments can be present in an amount ranging from 0.1% to 30% by weight, relative to the total weight of the corresponding composition, for example, ranging from 0.5% to 30%, such as ranging from 1 % to 20%, for instance ranging from 1 % to 15%, and ranging from 1 % to 10% by weight, relative to the total weight of the composition.

The particles of the at least one first and at least one second composite pigments can have a variety of forms. For example, the particles can be in at least one form chosen from flakes, globular, such as spherical, hollow, and solid forms. As used herein, the term “in the form of flakes” is understood to mean particles for which the ratio of the largest dimension to the thickness is greater than or equal to 5.

The at least one first and at least one second composite pigments of the present disclosure can, for example, have a specific surface area ranging from 1 m²/g (square meters/gram) to 1,000 m²/g, for instance, ranging from 10 m²/g to 600 m²/g, such as from 20 m²/g to 400 m²/g. As used herein, the term “specific surface area” is understood to mean the value measured using the BET (Brunauer-Emmett-Teller) method.

The core can be present in the at least one first and at least one second composite pigments in an amount greater than or equal to 50% by weight, relative to the total weight of the composite pigment, for example ranging from 50% to 70%, such as ranging from 60 to 70% by weight, relative to the total weight of the composite pigment.

The at least one first and/or at least one second compositions can comprise at least one composite pigment solely of the kind defined above or, alternatively, they can comprise at least one other composite pigment as well as pigments having a non composite structure, such as mineral pigments, interference pigments, lakes, or organic pigments. In one embodiment of the present disclosure, the at least one first and at least one second compositions do not comprise uncoated TiO₂ particles.

In one embodiment, the at least one first and at least one second composite pigments of the present disclosure are different from the an additional coloring agent or the at least one additional coloring substance that is adapted to produce a specific optical effect in the composition. Accordingly, in one embodiment of the present disclosure, the at least one first and at least one second composite pigments are not interference pigments.

Core

The cores of the at least one first and at least one second composite pigments, can be, for example, inorganic, and can have any form that is suitable for fixing particles of organic coloring substance. For example, non-limiting mention can be made of spherical, globular, granular, polyhedral, acicular, spindle-shaped, flattened in the form of a flake, a rice grain, scales, and a combination of these forms.

For example, the ratio of the largest dimension of the core to its smallest dimension can range from 1 to 50.

The core can have a mean size ranging from 1 nm (nanometer) to 100 nm, for instance, ranging from 5 nm to 75 nm, such as ranging from 10 nm to 50 nm. As used herein, the term “mean size” is understood to mean the dimension given by the statistical grain size distribution curve at 50% population, termed D50. The mean size may be a number average determined by image analysis (electron microscopy).

When the core is inorganic, is can have a refractive index greater than or equal to 2, such as greater than or equal to 2.1, for example greater than or equal to 2.2.

Among the materials that the core can be formed from, non-limiting mention can be made of metallic salts; metal oxides, such as oxides of titanium, zirconium, cerium, zinc, iron, iron blue, aluminum, and chromium; aluminas; glasses; ceramics; graphite; silicas; silicates for instance, aluminosilicates and borosilicates; synthetic mica; and mixtures thereof.

In one embodiment of the present disclosure, the core is made from oxides of titanium, such as TiO₂, of iron, such as Fe₂O₃, of cerium, zinc, aluminum, and silicates, such as aluminosilicates and borosilicates.

The core can have a specific surface area, measured using the BET method, ranging from 1 m²/g to 1,000 m²/g, such as ranging from 10 m²/g to 600 m²/g, for example ranging from 20 m²/g to 400 m²/g.

The core can be colored if desired.

Coloring Substance

The at least one first and at least one second coloring substances, when they are organic can, for example, comprise at least one organic pigment and/or at least one organic lake.

The at least one first and at least one second coloring substances can, for example, be selected from compounds that are insoluble in the physiologically acceptable medium of the composition.

The at least one first and at least one second coloring substances can, for example, comprise at least one pigment, for example, organic lakes, chosen from:
cochineal carmine;
the organic pigments of azo, anthraquinone, indigo, xanthene, pyrene, quinoline, triphenylmethane, and fluorane dyes;
organic lakes or organic insoluble salts of sodium, potassium, calcium, barium, aluminum, zirconium, strontium, titanium, and of acid dyes such as azo, anthraquinone, indigo, xanthene, pyrene, quinoline, triphenylmethane, fluorine dyes, and dyes comprising at least one group chosen from carboxylic and sulfonic acid group.

Among the organic pigments that may be used, non-limiting mention may be made of: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, and FD&C Yellow No. 6.

The at least one first and at least one second coloring substances can comprise at least one organic lake supported by an organic support such as colophane or aluminum benzoate, for example.

Among the organic lakes that may be used, for example, non-limiting mentioned may be made of: D&C Red No. 2 Aluminum lake, D&C Red No. 3 Aluminum lake, D&C Red No. 4 Aluminum lake, D&C Red No. 6 Aluminum lake, D&C Red No. 6 Barium lake, D&C Red No. 6 Barium/Strontium lake, D&C Red No. 6 Strontium lake, D&C Red No. 6 Potassium lake, D&C Red No. 7 Aluminum lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminum lake, D&C Red No. 9 Barium lake, D&C Red No. 9 Barium/Strontium lake, D&C Red No. 9 Zirconium lake, D&C Red No. 10 Sodium lake, D&C Red No. 19 Aluminum lake, D&C Red No. 19 Barium lake, D&C Red No. 19 Zirconium lake, D&C Red No. 21 Aluminum lake, D&C Red No. 21 Zirconium lake, D&C Red No. 22 Aluminum lake, D&C Red No. 27 Aluminum lake, D&C Red No. 27 Aluminum/Titanium/Zirconium lake, D&C Red No. 27 Barium lake, D&C Red No. 27 Calcium lake, D&C Red No. 27 Zirconium lake, D&C Red No. 28 Aluminum lake, D&C Red No. 30 lake, D&C Red No. 31 Calcium lake, D&C Red No. 33 Aluminum lake, D&C Red No. 34 Calcium lake, D&C Red No. 36 lake, D&C Red No. 40 Aluminum lake, D&C Blue No. 1 Aluminum lake, D&C Green No. 3 Aluminum lake, D&C Orange No. 4 Aluminum lake, D&C Orange No. 5 Aluminum lake, D&C Orange No. 5 Zirconium lake, D&C Orange No. 10 Aluminum lake, D&C Orange No. 17 Barium lake, D&C Yellow No. 5 Aluminum lake, D&C Yellow No. 5 Zirconium lake, D&C Yellow No. 6 Aluminum lake, D&C Yellow No. 7 Zirconium lake, D&C Yellow No. 10 Aluminum lake, FD&C Blue No. 1 Aluminum lake, FD&C Red No. 4 Aluminum lake, FD&C Red No. 40 Aluminum lake, FD&C Yellow No. 5 Aluminum lake, and FD&C Yellow No. 6 Aluminum lake.

The chemical compounds corresponding to each of the coloring substances listed above are mentioned in the work entitled “International Cosmetic Ingredient Dictionary and Handbook ”, 1997 edition, pages 371 to 386 and 524 to 528, published by “The Cosmetic, Toiletry, and Fragrance Association,” the contents of which are hereby incorporated by reference.

The at least one first and at least one second organic coloring substances of the at least one first and/or at least one second composite pigments can be present in an amount ranging from 10 parts to 500 parts by weight per 100 parts of core, such as ranging from 20 parts to 250 parts by weight, for example ranging from 40 parts to 125 parts by weight per 100 parts of core.

The at least one organic coloring substance in the at least one composite pigment can be present in an amount greater than or equal to 30% by weight, relative to the total weight of the composite pigment, for example ranging from 30% to 50%, such as from 30% to 40% by weight, relative to the total weight of the composite pigment.

Binder

The at least one optional binder may be of any type provided that it allows the at least one first and/or at least one second organic coloring substances to adhere to the surface of the core.

For example, the at least one binder can be chosen from, by way of non-limiting examples, silicone compounds, polymeric, oligomeric and similar compounds, such as organosilanes, fluoroalkylated organosilanes and polysiloxanes, for example polymethylhydrogen siloxane, as well as to variety of coupling agents such as coupling agents based on silanes, titanates, aluminates, zirconates, and mixtures thereof.

Non-limiting examples of the silicone compounds that can be used include:
organosilanes (1) obtained from alkoxysilanes;
polysiloxanes (2) which can optionally be modified, chosen from:
modified polysiloxanes (2A) comprising at least one radical chosen for instance from polyethers, polyesters and epoxy compounds (termed “modified polysiloxanes” below);
polysiloxanes (2B) carrying, on one silicon atom located at the end of the polymer, at least one group chosen from the following non-limiting list: carboxylic acids, alcohols, and hydroxyl groups; and
fluoroalkylated organosilane compounds (3) obtained from fluoroalkylsilanes.

The organosilane compounds (1) can be obtained from alkoxysilane compounds of formula(I):
R¹ₐSiX₄₋ₐ (I)
wherein:
R¹ is chosen from C₆H₅—, (CH₃)₂CHCH₂— and —C_{b}H_{2b+1}— radicals (in which b ranges from 1 to 18);
X is chosen from CH₃O— and C₂H₅O— radicals; and
a ranges from 0 to 3.

Non-limiting examples of alkoxysilane compounds that may be used include alkoxysilanes chosen from: methyltriethoxysilane, dimethyldiethoxysilane, phenyltriethoxysilane, diphenyldiethoxysilane, methyltrimethoxysilane, dimethyldimethoxysilane, phenyltrimethoxysilane, diphenyldimethoxysilane, isobutyltrimethoxysilane, decyltrimethoxysilane, and the like. For example, in one embodiment of the present disclosure, the alkoxysilane compounds are chosen from methyltriethoxysilane, phenyltriethoxysilane, methyltrimethoxysilane, dimethyidimethoxysilane, isobutyltrimethoxysilane, such as from methyltriethoxysilane, methyltrimethoxysilane, and phenyltriethoxysilane.

The polysiloxanes (2) can be chosen from those of formula (II): 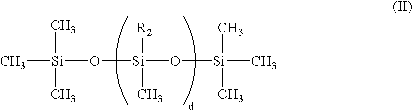
wherein R² is chosen from a hydrogen atom and CH₃— radicals and d ranges from 15 to 450.

In one embodiment of the present disclosure, R² is chosen from hydrogen atoms.

The modified polysiloxanes (2A) can also be chosen from
(a¹) modified polysiloxanes carrying polyethers, chosen from those of formula (III): 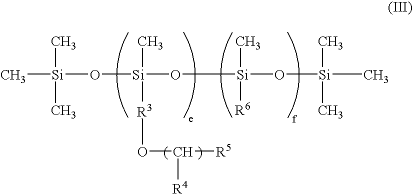
   wherein R³ is chosen from —(CH₂)ₕ— radicals; R⁴ is chosen from —(CH₂)ᵢ—CH₃ radicals; R⁵ is chosen from —OH, —COOH, —CH═CH₂, —C(CH₃)═CH₂ and —(CH₂)ⱼ—CH₃ radicals; R⁶ is chosen from —(CH2)ₖ—CH₃ radicals; g and h, independently of each other, range from 1 to 15; j and k, independently of each other, range from 0 to 15; e ranges from 1 to 50, and f ranges from 1 to 300;
(a²) modified polysiloxanes carrying polyesters, chosen from those of formula (IV): 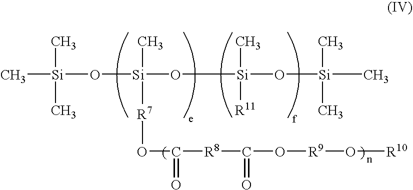
   wherein R⁷, R⁸ and R⁹, which may be identical or different, are chosen from —(CH₂)_{q}— radicals; R¹⁰ is chosen from —OH, —COOH, —CH═CH₂, —C(CH₃)═CH₂ and —(CH₂)ᵣ—CH₃ radicals; R¹¹ is chosen from —(CH₂)ₛ—CH₃ radicals; n and q, independently of each other, range from 1 to 15, r and s, independently of each other, range from 0 to 15; e ranges from 1 to 50, and f ranges from 1 to 300; and
(a³) modified polysiloxanes carrying epoxy radicals, chosen from those of formula (V): 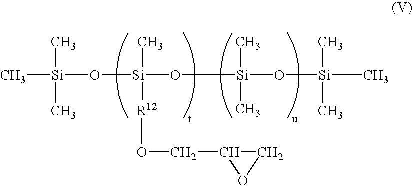
   wherein R¹² is chosen from —(CH₂)ᵥ— radicals; v ranges from 1 to 15; t ranges from 1 to 50, and u ranges from 1 to 300.

In one embodiment of the present disclosure, the polysiloxanes (2A) are modified polysiloxanes carrying polyethers with formula (III).

Polysiloxanes modified at the terminal portion (2B) can also be chosen from those of formula (VI): 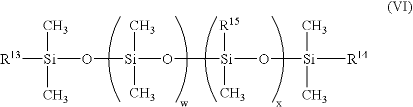
wherein R¹³ and R¹⁴, which may be identical or different, are chosen from —OH groups, and R¹⁶—OH, and R¹⁷—COOH radicals; R¹⁵ is chosen from —CH₃ and —C₆H₅ radicals; R¹⁶ and R¹⁷ are chosen from —(CH₂)_{y}— radicals; y ranges from 1 to 15; w ranges from 1 to 200; and x ranges from 0 to 100.

In one embodiment of the present disclosure, the polysiloxanes modified on at least one end are chosen from those carrying at least one radical (R¹⁶ and/or R¹⁷) carrying a carboxylic acid group on at least one terminal silicon atom.

Fluoroalkylated organosilane compounds (3) can also be chosen from fluoroalkylsilanes of formula (VII):
CF₃(CF₂)_{z}CH₂CH₂(R¹⁸)ₐSiX₄₋ₐ (VII)
wherein:
R¹⁸ is chosen from CH₃—, C₂H₅—, CH₃O—, and C₂H₅O— radicals;
X is chosen from CH₃O— and C₂H₅O— radicals;
z ranges from 0 to 15 and a ranges from 0 to 3.

By way of non-limiting example, the fluoroalkylsilanes may be chosen from trifluoropropyltrimethoxysilane, tridecafluorooctyltrimethoxysilane, heptadecafluorodecyltrimethoxysilane, heptadecafluorodecylmethyldimethoxysilane, trifluoropropyltriethoxysilane, tridecafluorooctyltriethoxysilane, heptadecafluorodecyltriethoxysilane, heptadecafluorodecylmethyldiethoxysilane and the like. In one embodiment of the present disclosure, the fluoroalkylsilanes are chosen from trifluoropropyltrimethoxysilane, tridecafluorooctyltrimethoxysilane and heptadecafluorodecyltrimethoxysilane, such as trifluoropropyl trimethoxysilane and tridecafluorooctyltrimethoxysilane.

Among the silane-based coupling agents that can be used, non-limiting mention may be made of vinyltrimethoxysilane, vinyltriethoxysilane, γ-aminopropyl-triethoxysilane, γ-glycidoxypropyltrimethoxysilane, γ-mercaptopropyltrimethoxysilane, γ-methacryloxypropyltrimethoxysilane, N-β(aminoethyl)-γ-aminopropyltrimethoxysilane, γ-glycidoxypropylmethyldimethoxysilane, γ-chloropropyltrimethoxysilane, and the like.

The titanate-based coupling agents can be chosen from: isopropylstearoyl titanate, isopropyltris(dioctylpyrophosphate) titanate, isopropyltri(N-aminoethyl-aminoethyl) titanate, tetraoctylbis(ditridecylphosphate) titanate, tetra(2,2-diaryloxymethyl-1-butyl)bis(ditridecyl)phosphate titanate, bis(dioctylpyrophosphate)oxyacetate titanate, bis(dioctylpyrophosphate)ethylene titanate, and the like.

The aluminate-based coupling agents can be chosen from acetoalkoxyaluminum diisopropylate, aluminum diisopropoxymonoethylacetoacetate, aluminum trisethylacetoacetate, aluminum trisacetylacetonate, and the like.

The zirconate-based coupling agents can be chosen from zirconium tetrakisacetylacetonate, zirconium dibutoxybisacetylacetonate, zirconium tetrakisethylacetoacetate, zirconium tributoxymonoethylacetoacetate, zirconium tributoxyacetylacetonate, and the like.

The at least one binder can be chosen from compounds having a molar mass ranging from 300 to 100,000.

To obtain a layer which uniformly coats the inorganic cores, the binder can be, for example, in the liquid state or can be soluble in water or other solvents.

The at least one binder, when present, can be present in an amount ranging from 0.01% to 15%, such as from 0.02% to 12.5%, for example, from 0.03% to 10% by weight (calculated with respect to C or Si) relative to the weight of the at least one first and at least one second particles comprising the core and the binder. Further details regarding the calculation of the relative quantity of binder can be found in European Patent Application No. EP 1 184 426 A2.

The at least one binder, when present, can be present in an amount less than or equal to 5%, such as less than or equal to 3% by weight, relative to the total weight of the at least one first and/or at least one second composite pigment.

Preparation of the First and Second Composite Pigments

The at least one first and at least one second composite pigments can be manufactured by any appropriate method, for example a mechano-chemical method or a method of precipitation in solution, with dissolution of an organic coloring substance and a precipitation thereof at the surface of the core.

At least one binder may or may not be used.

For example, a method comprising a mechanical mixing of at least one organic pigment and an inorganic core can be used. A binder may be added and mixed with the inorganic core before the introduction of the at least one organic coloring substance.

The at least one first and at least one second composite pigments can, for example, be produced using one of the processes described in European Patent Application Nos. EP 1 184 426 and EP 1 217 046, the contents of which are hereby incorporated by reference. For example, in one embodiment of the present disclosure, the process described in EP 1 184 426 is used.

In another embodiment of the present disclosure, the at least one first and at least one second particles intended to constitute the core are first mixed with at least one binder.

So that the binder can adhere uniformly to the surface of the core, the particles can, for example, pass initially through a mill to disaggregate them.

The mixing and agitation conditions are selected so that the core is uniformly coated with binder. Such conditions can be controlled so that the linear load ranges from 19.6 N/cm (newtons/centimeter) to 19160 N/cm, for example, ranging from 98 N/cm to 14170 N/cm, such as ranging from 147 N/cm to 980 N/cm; the period of treatment time ranges from 5 minutes to 24 hours, such as from 10 minutes to 20 hours; the rotation rate can range from 2 rpm (revolutions per minute) to 1,000 rpm, for instance, ranging from 5 rpm to 1,000 rpm, such as from 10 rpm to 800 rpm.

After coating the core with at least one binder, the at least one coloring substance is added and mixed with agitation so that it adheres to the layer of binder.

Examples of addition methods include continuous addition in large quantities, or in small quantities.

Mixing and agitation, whether of the cores with the binder, or of the coloring substance with the cores coated with binder, can be carried out using an apparatus which can apply a sharp shearing and/or compressive force to the mixture of powders. Examples of apparatus of that type are roller mixers, blade mixers, and the like. In one embodiment of the present disclosure, a roller mixer is used. A list of suitable apparatus is given in European Patent Application No. EP 1 184 426 A2.

A further method for manufacturing the at least one first and at least one second composite pigments has been described in Japanese Patent No. JP 3286463, which discloses a solution precipitation process. The at least one coloring substance is dissolved in ethanol and the cores are then dispersed in said ethanolic solution. An aqueous alkaline solution of sodium or potassium carbonate is then slowly added to these mixtures and finally, an ethanolic calcium chloride solution is slowly added, with constant agitation.

Other Components

Oil

The at least one first and at least one second compositions of the present disclosure can further comprise, for example, at least one oil. The at least one oil can be chosen from volatile oils, and non-volatile oils.

As used herein, the term “volatile oil” is understood to mean any oil that is liable to evaporate on contact with the skin at ambient temperature and at atmospheric pressure. Volatile oils of the present disclosure are volatile cosmetic oils, that are liquid at ambient temperature, having non-zero vapor pressure at ambient temperature and atmospheric pressure, for example, vapor pressure ranging from 0.13 Pascals (Pa) to 40,000 Pa (0.001 millimetres of mercury (mmHg) to 300 mmHg), such as ranging from 1.3 Pa to 1300 Pa (0.01 mmHg to 10 mmHg).

As used herein, the term “non-volatile” oil is understood to mean an oil that remains on the skin at ambient temperature and atmospheric pressure, at least for several hours, and for instance, has vapor pressure of less than 0.13 Pa (0.001 mmHg).

Such volatile or non-volatile oils may be hydrocarbon oils, such as of animal or vegetable origin, silicone oils, or mixtures thereof. As used herein, the term “hydrocarbon oil” is understood to mean an oil comprising mainly atoms of hydrogen and carbon and optionally atoms of oxygen, nitrogen, sulfur, and/or phosphorous.

The volatile hydrocarbon oils can be chosen from hydrocarbon oils having 8 to 16 atoms of carbons, for instance branched C₈-C₁₆ alkanes such as C₈-C₁₆ isoalkanes of petroleum origin (also known as isoparaffins) such as isododecane (also known as 2,2,4,6,-pentamethylheptane), isodecane, and isohexadecane, e.g. the oils sold under the trade names Isopars or Permethyls.

As volatile oils, it is also possible to use volatile silicones, such as, for example volatile linear or cyclic silicone oils, for instance, those having a viscosity less than or equal to 5 centistokes (5×10⁻⁶ square meters per second (m²/s)), and for instance, having from 2 to 10 atoms of silicon, such as from 2 to 7 atoms of silicon, such silicones optionally including alkyl or alkoxy groups having from 1 to 10 atoms of carbon. Volatile silicone oils suitable for use in the present disclosure include, by way of non-limiting example octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane, and mixtures thereof.

The at least one volatile oil, when present, can be present in the at least one first and/or at least one second compositions in an amount ranging from 0.1% to 98% by weight, for instance ranging from 1% to 65% by weight, such as ranging from 2% to 50% by weight, relative to the total weight of the composition.

The non-volatile oils can be chosen, for example, from optionally-fluorinated hydrocarbon oils and/or non-volatile silicone oils.

Non-limiting examples of non-volatile hydrocarbon oils that can be mentioned include:
hydrocarbon oils of animal origin;
hydrocarbon oils of vegetable origin such as triglyercides constituted by esters of fatty acids and glycerol in which the fatty acids may have various chain lengths ranging from C₄ to C₂₄, which chains may be linear or branched, saturated or unsaturated; for instance, these oils may be triglycerides of heptanoic acid or of octanoic acid, or oils of wheat germ, sunflower, grapeseed, sesame seed, corn (maize), apricot kernel, castoroil, shea, avocado stone, olive, soybean, sweet almond, palm, rape-seed, cotton, hazelnut, macadamia nut, jojoba, alfalfa, poppy, Hokkaido pumpkin, sesame, squash, black current, evening primrose, millet, barley, quinoa, rye, safflower, candlenut, passion fruit, muscat rose; shea butter; or indeed triglycerides of caprylic/capric acids such as those sold by the supplier STEARINERIES DUBOIS or those sold under the names MIGLYOL 810®, 812®, and 818® by the supplier DYNAMIT NOBEL;
   synthetic ethers having from 10 to 40 carbons atoms;
   linear or branched hydrocarbons of mineral or synthetic origin such as petroleum jelly, polydecenes, hydrogenated polyisobutene such as Parleam®, squalane, paraffin oils, and mixtures thereof;
   synthetic esters such as oils of formula R1COOR2, wherein R1 is chosen from the remainder of a linear or branched fatty acids having from 1 to 40 carbon atoms, and R₂ is chosen from hydrocarbon chains, such as a branched chain, comprising from 1 to 40 carbon atoms, with the condition that R₁+R₂ is greater than or equal to 10, such as, for example, Purcellin oil (cetostearyle octanoate), isopropyl myristate, isopropyl palmitate, C₁₂ to C₁₅ alcohol benzoates, hexyl laurate, diisopropyl adipate, isononyl isononanoate, 2-ethyl-hexyl palmitate, isotearyl isostearate, heptanoates, octanoates, decanoates or ricinoleates of alcohols or of polyalcohols such as propylene glycol dioctanoate; hydroxylated esters such as isostearyl lactate, di-isostearyl malate, polyol esters, and pentaerythritol esters;
   fatty acids liquid at ambient temperature having a branched and/or unsaturated carbon chain with 12 to 26 carbon atoms such as octyl dodecanol, isostearylic alcohol, oleic alcohol, 2-hexyldecanol, 2-butyloctanol, and 2-undecylpentadecanol; and
   higher fatty acids such as oleic acid, linoleic acid, linolenic acid, and mixtures thereof.

The at least one non-volatile silicone oil that can be used in the at least one first and/or at least one second compositions of the set of the present disclosure can be chosen from non-volatile polydimethylsiloxanes (PDMS), polydimethylsiloxanes including alkyl or alkoxy groups that are hanging and/or at the ends of the silicone chain, groups each having from 2 to 24 carbon atoms, phenyl silicones such as phenyl trimethicones, phenyl dimethicones, phenyl trimethylsiloxy diphenylsiloxanes, diphenyl dimethicones, and diphenyl methyldiphenyl trisiloxanes.

The at least one non-volatile oil, when present, can be present in the at least one and/or at least one second composition of the present disclosure in an amount ranging from 0.1% to 98% by weight, such as ranging from 0.1% to 85% by weight, for instance, ranging from 1% to 70% by weight, relative to the total weight of the composition.

Oils can be present in an amount, when they are present in the at least one first and/or at least one second compositions, ranging from 0.01% to 99% by weight, relative to the total weight of the composition, for instance, from 0.05% to 60% by weight, such as from 1% to 75% by weight, relative to the total weight of the composition.

Wax

The least one first and/or at least one second compositions of the present disclosure may include at least one wax. The term “wax” is used in the present invention to mean a lipophilic compound that is solid at ambient temperature (25° C.), that presents a reversible solid/liquid change of state, with a melting point greater than or equal to 30° C., and possibly being as high as 200° C.

The melting point of wax can be measured using differential scanning calorimeter (DSC), for example the calorimeter sold under the name DSC 30 by the supplier METLER.

The waxes may be hydrocarbons, fluorinated, and/or silicone waxes, and they may be of vegetable, mineral, animal, and/or synthetic origin. The waxes, for example, can have a melting temperature greater than or equal to 30° C., such as greater than or equal to 45° C.

Waxes suitable for use in the at least one first and/or at least one second compositions-of the present disclosure include, by way of non-limiting example: bees wax, caranda or candellila wax, paraffin, microcrystalline waxes, ceresin, or ozocerite; synthetic waxes such as polyethylene waxes or Fischer Tropsch waxes, silicone waxes such as alkyl or alkoxy-dimethicone having 16 to 45 carbon atoms.

For example, the at least one first and/or at least one second compositions can comprise at least one wax in an amount ranging from 1% to 50% by weight, relative to the total weight of the composition, such as from 5% to 30% by weight.

Fillers

The at least one first and/or at least one second compositions of the present disclosure may further comprise at least one filler, for example, in an amount ranging from 0.01% to 50% by weight, relative to the total weight of the composition, such as ranging from 0.01% to 30% by weight, and from 0.01% to 10% by weight. As used herein, the term “fillers” is understood to mean particles of any form whether colorless or white, of mineral or synthetic origin, that are insoluble in the medium of the composition regardless of the temperature at which the composition is made. These fillers serve for example, to modify the rheology or the texture of the composition.

The at least one filler can be chosen from inorganic and organic fillers having any shape, being in the form of plates, spheres, and oblong, and regardless of their crystallographic form (e.g. flakes, cubic, hexagonal, orthorombic, etc.). Among the fillers that can be used according to the present disclosure, non-limiting mention can be made of talc, mica, silica, kaolin, powders of polyamide (Nylon®) (Orgasol® from ATOCHEM), poly-β-alanine and polyethylene, powders of tetrafluoroethylene polymers (Teflon®), lauroyl-lysine, starch, boron nitride, hollow polymer microspheres such as those made of polyvinylidene chloride and acrylonitrile such as Expancel® (NOBEL INDUSTRIE), copolymers of acrylic acid (Polytrap® from DOW CORNING) and microbeads of silicone resin (Tospearls® from TOSHIBA, for example), particles of elastomer polyorganosiloxanes, precipitated calcium carbonate, magnesium carbonate and hydro-carbonate, hydroxyapatite, hollow silica microspheres (Silica Beads® from MAPRECOS), microcapsules of glass or of ceramic, metallic soaps derived from carboxylic organic acids having from 8 to 22 carbon atoms, such as from 12 to 18 carbon atoms, e.g. zinc, magnesium, or lithium stearate, zinc laurate, or magnesium myristate.

Additional Coloring Substance

The at least one first and/or at least one second compositions in the set as disclosed herein may include at least one first and/or at least one second additional coloring substance which differs from the at least one first and at least one second composite pigments as disclosed herein.

The at least one first and/or at least one second additional coloring substances can be chosen from mineral pigments, organic pigments, pearlescent pigments, liposoluble colorants, and hydrosoluble colorants.

The mineral pigments can be white or colored, and may optionally be coated. By way of non-limiting examples of mineral pigments, mention may be made of titanium dioxide, which may be surface treated; oxides of zirconium or cerium; and oxides of iron or of chromium; manganese violet; ultramarine blue; chromium hydrate; and iron blue. The mineral pigments may be present in an amount ranging from 0% to 40%, such as from 1% to 35%, and from 2% to 25% by weight, relative to the total composition weight.

The pearlescent pigments can be chosen from white pearlescent pigments such as titanium-covered mica, or bismuth oxychloride, colored pearlescent pigments such as titanium mica with iron oxides, titanium mica for instance, with ferric blue or with chromium oxide, titanium mica with an organic pigment of the above-specified type, and pearlescent pigments based on bismuth oxychloride. The pearlescent pigments may be present in an amount ranging from 0% to 20% by weight, relative to the total weight of the composition, such as from 0.1% to 15% (when present).

Among the organic pigments that may be used, non-limiting mention may be made of carbon black, D&C type pigments, and lakes based on carmine cochineal, barium, strontium, calcium, or aluminum.

Non-limiting examples of liposoluble colorants include Sudan red, D&C Red No. 17, D&C Green No. 6, β-carotene, soybean oil, Sudan brown, D&C Yellow No. 11, D&C Violet No. 2, D&C orange No. 5, and quinoline yellow.

Non-limiting examples of hydrosoluble colorants include beetroot juice and methylene blue.

The colorants, when present, may be present in an amount ranging from 0.1% to 20% by weight, relative to the weight of the composition, such as from 0.1% to 6% (if present).

Other Ingredients

For example, the at least one first and/or at least one second compositions, in the set, can be an anhydrous composition, i.e. a composition comprising less than 2% by weight of water, for instance less than 0.5% of water, such as comprising no water, with any water present not being added during preparation of the composition, but corresponding to residual water brought in by the mixed ingredients.

The at least one first and/or at least one second compositions can further comprise at least one other conventional cosmetic ingredient chosen from, for example, antioxidizers, film-generating polymers, fragrances, preservatives, thickeners such as bentone, neutralizing agents, wetting agents, sunscreens, vitamins, moisturizing agents, and cosmetic and/or dermatological active agents.

Naturally, the person skilled in the art will ensure that such optional additional ingredients are selected and/or present in quantities suitable for ensuring that the beneficial properties of the set of at least two solid compositions of the present disclosure are not, or not substantially, degraded by such an addition.

The set of at least two solid makeup compositions of the present disclosure can be compositions for making up the skin or the lips, such as foundations, eye shadow, blushers, concealers of rings and/or bags under the eyes, body makeup, and lipsticks. In one embodiment of the present disclosure, the set of at least two solid makeup compositions is a set of lipsticks.

Throughout the description, the term “comprising a” should be understood as being synonymous with “comprising at least one” unless specified to the contrary.

The ranges given include the limit values, unless stated to the contrary.

Although the present disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present invention as defined by the appended claims.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, the numerical values set forth in the specific example are reported as precisely as possible. Any numerical value, however, inherently contains certain errors necessarily resulting from the standard deviation found in their respective testing measurements.

The following examples are intended to illustrate the present disclosure in a non-limiting manner.

### EXAMPLES

Several lipsticks were made in the form of sticks having the following composition (quantities expressed in % by weight relative to the total weight of the composition).

| | |
|---|---|
| Octyl-2 dodecanol | 15.63% |
| Ditertiobutyl 4-hydroxytoluene | 0.06% |
| Isopropyl lanolate | 9.59% |
| Triglycerides of lauric/palmitic/cetylic/stearic | 5% |
| acids in the proportions 50/20/10/10 | |
| (Softisan 100 from SASOL) | |
| Protected acetylated lanolin | 9.59% |
| Phenyl trimethylsiloxy trisiloxane | 4.26% |
| Polyethylene wax (MW: 500) | 8.8% |
| (Polywax 500 from BARECO) | |
| Di-iso-strearyl malate | 13.07% |
| Liquid lanolin9.60 | |
| Tri-decyl tri-mellitate | 10.40% |
| Microcrystalline wax | 4% |
| Coloring substance under test | 10% |

All of the tested compositions had the above basic formulation and they differed in the nature of the coloring substance, which substance was present in an amount of 10% by weight.

### Examples 1, 2, and 3

### In Accordance with the Present Disclosure

Lipstick Example 1 included a composite pigment of the present disclosure.

The coloring substance used in Example 1 was a composite pigment constituted by 50 parts by weight of organic pigment known as D&C Red No. 7 per 100 parts of an inorganic core of titanium dioxide having a mean size of 20 nm and a specific surface area of 50 m²/g, and made using 1.3 parts of polymethyl hydrogen siloxane binder.

The lipstick of Example 2 comprised a composite pigment of the present disclosure constituted by 50 parts by weight of an organic pigment known as FD&C Yellow No. 5 per 100 parts of an inorganic core of titanium dioxide having a mean size of 20 nm and a specific surface area of 50 m²/g, and made with 1.3 parts of a polymethyl hydrogen siloxane binder.

The lipstick of Example 3 in accordance with the present disclosure comprised a composite pigment comprising 70 parts by weight of the organic lake known as FD&C Blue 1 Al lake per 100 parts of an inorganic titanium dioxide core of mean size of 20 nm and of specific surface area 50 m²/g, and made with a polymethyl hydrogen siloxane binder.

Examples 1, 2, and 3 constitute a set of lipsticks of different colors. The hardness of the sticks obtained with those three compositions was measured using the method described above.

The hardness measured for the stick of Example No. 1 was 149 g (i.e. 1.46 N) y.

The hardness measured for the stick of Example No. 2 was 167 g (i.e. 1.636 N).

The hardness measured for the stick of Example No. 3 was 163 g (i.e. 1.597 N).

Thus, the maximum difference in hardness for those three compositions within the same set was 18 g (i.e. 0.176 N).

### Comparative Examples 1, 2, and 3

Three compositions were made comprising the above-described basic formulation and incorporating the following coloring substances: for comparative Example No. 1, an organic pigment known as D&C Red No. 7, for comparative Example No. 2, an organic pigment known as FD&C Yellow No. 5, and for comparative Example No. 3, an organic lake known as FD&C Blue 1 Al lake, with the three coloring substances being present in an amount of 10% by weight of each of the compositions.

For comparative Example No. 1, the measured hardness was 117 g (i.e. 1.146 N).

For comparative Example No. 2, the measured hardness was 166 g (i.e. 1.626 N).

For comparative Example No. 3, the measured hardness was 155 g (i.e. 1.519 N).

A relatively large difference in hardness was thus obtained of the order of 49 g (i.e. 0.48 N) for this range of sticks constituted by comparative Examples Nos.1, 2, and 3.

### Comparative Examples 4, 5, and 6

Three other compositions were made in the form of lipstick sticks incorporating 10% by weight of the following coloring substances.

Comparative Example No. 4 comprised a mixture of nano TiO₂ (6.21%) and of the organic pigment D&C Red No. 7 (3.29%).

Comparative Example No. 5 comprised a mixture of nano TiO₂ (6.21%) and of the organic pigment FD&C Red No. 5 (3.29%).

Comparative Example No. 6 comprised a mixture of nano TiO₂ (5.93%) and of the organic laque FD&C Blue 1 Al lake (4.07%).

The measured hardnesses were as follows:

For comparative Example No. 4, the measured hardness was 106 g (i.e. 1.039 N).

For comparative Example No. 5, the measured hardness was 141 g (i.e. 1.382 N).

For comparative Example No. 6, the measured hardness was 143 g (i.e. 1.401 N).

Thus the difference in hardness between these three compositions in a given range of sticks was 37 g (i.e. 0.36 N).

The tests performed show that the compositions of Examples 1, 2, and 3 forming a set of at least two solid compositions in accordance with the present disclosure comprising composite pigments presented hardness differences of less than 20 g (i.e. 0.196 N), in spite of the different natures of the coloring substances in the composite pigments.

1. A set of at least two solid makeup compositions for application to skin, lips, nails, or hair, comprising:
at least one first composition having a first color due at least in part to the presence of at least one first composite pigment; and
at least one second composition having a second color different from the first color, due at least in part to the presence of at least one second composite pigment different from the at least one first composite pigment;
wherein the at least one first and at least one second composite pigments comprise particles comprising a core, which is at least partially coated with at least one coloring substance;
with the proviso that the coloring substances of the at least one first and at least one second composite pigments are different, and
wherein the difference in hardness between the at least one first and at least one second compositions is less than or equal to 25 g (0.245 N).

2. The set of claim 1, wherein the cores of the at least one first and at least one second composite pigments are such that the difference in hardness between the at least one first and at least one second compositions is less than or equal to 25 g (0.245 N).

3. The set of at least two solid makeup compositions according to claim 1, wherein the at least one first and at least one second composite pigments have cores of the same material.

4. The set of at least two solid makeup compositions according to claim 1, comprising at least three compositions, wherein the difference in hardness between the at least three compositions is less than or equal to 25 g (0.245 N), and
with the proviso that the coloring substances of the at least one first, at least one second, at least one third composite pigments are different.

5. The set of at least two solid makeup compositions according to claim 3, comprising at least five compositions, wherein the difference of hardness between the at least five compositions is less than or equal to 25 g (0.245 N), and
with the proviso that the coloring substances of the at least one first, at least one second, at least one third, at least one fourth, and at least one fifth composite pigments are different.

6. The set of at least two solid makeup compositions according to claim 1, wherein the cores of the at least one first and at least one second composite pigments are inorganic.

7. The set of at least two solid makeup compositions according to claim 1, wherein the cores of the at least one first and at least one second composite pigments are organic.

8. The set of at least two solid makeup compositions according to claim 1, wherein the coloring substance coatings of the at least one first and at least one second composite pigments are an organic coloring substance.

9. The set of at least two solid makeup compositions according to claim 1, wherein the compositions are for application to the lips.

10. The set of at least two solid makeup compositions according to claim 1, wherein the composition is in the form of a stick.

11. The set of at least two solid makeup compositions according to claim 9, wherein the at least one first and at least one second compositions are packaged with mechanisms comprising at least two portions that can turn relative to each other in order to move the sticks.

12. The set of at least two solid makeup compositions according to claim 1, wherein the at least one first and at least one second compositions comprise the same ingredients apart from the at least one first and at least one second composite pigments.

13. The set of at least two solid makeup compositions according to claim 1, wherein the at least one first and at least one second compositions further comprise at least one solvent, wherein the at least one solvent is the same for the at least one first and at least one second compositions.

14. The set of at least two solid makeup compositions according to claim 1, wherein the at least one first and at least one second compositions further comprise at least one wax, wherein the at least one wax is the same for the at least one first and at least one second compositions.

15. The set of at least two solid makeup compositions according to claim 1, wherein the hardness difference between the at least one first and at least one second compositions is less than or equal to 20 g (0.196 N).

16. The set of at least two solid makeup compositions according to claim 1, wherein the at least one first and at least one second compositions do not comprise coloring substances other than the at least one first and at least one second composite pigments.

17. The set of at least two solid makeup compositions according to claim 1, wherein at least one of the at least one first and at least one second compositions comprises at least one additional coloring substance different from the at least one first and at least one second composite pigments, wherein the at least one first and/or at least one second composite pigments are present in an amount greater than or equal to the amount of the at least one additional coloring substance.

18. The set of at least two solid makeup compositions according to claim 1, wherein the at least one first and/or at least one second composite pigments are present in an amount ranging from 0.1% to 30% by weight, relative to the total weight of the at least one first and/or at least one second compositions.

19. The set of at least two solid makeup compositions according to claim 17, wherein the at least one first and/or at least one second composite pigments are present in an amount ranging from 0.5% to 30% by weight, relative to the total weight of the at least one first and/or at least one second compositions.

20. The set of at least two solid makeup compositions according to claim 18, wherein the at least one first and/or at least one second composite pigments are present in an amount ranging from 1% to 20% by weight, relative to the total weight of the at least one first and/or at least one second compositions.

21. The set of at least two solid makeup compositions according to claim 19, wherein the at least one first and/or at least one second composite pigments are present in an amount ranging from 1% to 15% by weight, relative to the total weight of the at least one first and/or at least one second compositions.

22. The set of at least two solid makeup compositions according to claim 21, wherein the at least one first and/or at least one second composite pigments are present in an amount ranging from 1% to 10% by weight, relative to the total weight of the at least one first and/or at least one second compositions.

23. The set of at least two solid makeup compositions according to claim 1, wherein the cores of the at least one first and/or at least one second composite pigments each have a mean size ranging from 1 nm to 100 nm.

24. The set of at least two solid makeup compositions according to claim 23, wherein the cores of the at least one first and/or at least one second composite pigments each have a mean size ranging from 5 nm to 75 nm.

25. The set of at least two solid makeup compositions according to claim 24, wherein the cores of the at least one first and/or at least one second composite pigments each have a mean size ranging from 10 nm to 50 nm.

26. The set of at least two solid makeup compositions according to claim 1, wherein the cores of the at least one first and/or at least one second composite pigments each have a specific surface area ranging from 10 m²/g to 1,000 m²/g.

27. The set of at least two solid makeup compositions according to claim 26, wherein the cores of the at least one first and/or at least one second composite pigments each have a specific surface area ranging from 10 m²/g to 600 m²/g.

28. The set of at least two solid makeup compositions-according to claim 27, wherein the cores of the at least one first and/or at least one second composite pigments each have a specific surface area ranging from 20 m²/g to 400 m²/g.

29. The set of at least two solid makeup compositions according to claim 1, wherein the cores of the at least one first and/or at least one second composite pigments are in the forms chosen from spherical, globular, polyhedral, acicular, spindle-shaped, and flattened forms.

30. The set of at least two solid makeup compositions according to claim 1, wherein the cores of the at least one first and/or at least one second composite pigments comprise at least one material chosen from metallic salts, metal oxides, alumina, glass, ceramics, graphite, silica, silicates, and synthetic micas.

31. The set of at least two solid makeup compositions according to claim 30, wherein the cores of the at least one first and/or at least one second composite pigments comprise a metal oxide chosen from an oxide of titanium, zirconium, cerium, zinc, iron, iron blue, chromium and aluminum.

32. The set of at least two solid makeup compositions according to claim 31, wherein the metal oxide is chosen from an oxide of titanium, iron, cerium, zirconium, zinc, or aluminum.

33. The set of at least two solid makeup compositions according to claim 32, wherein the metal oxide is titanium dioxide.

34. The set of at least two solid makeup compositions according to claim 30, wherein the cores of the at least one first and/or at least one second composite pigments comprise at least one silicate chosen from an aluminosilicate and a borosilicate.

35. The set of at least two solid makeup compositions according to claim 30, wherein the cores of the at least one first and/or at least one second composite pigments comprise silica.

36. The set of at least two solid makeup compositions according to claim 1, wherein the at least one first and/or at least one second organic coloring substances of the at least one first and/or at least one second composite pigments are present in an amount ranging from 10 to 500 parts by weight, per 100 parts by weight of the core.

37. The set of at least two solid makeup compositions according to claim 36, wherein the at least one first and/or at least one second coloring substances of the at least one first and/or at least one second composite pigments are present in an amount ranging from 20 to 250 parts by weight, per 100 parts by weight of the core.

38. The set of at least two solid makeup compositions according to claim 37, wherein the at least one first and/or at least one second coloring substances of the at least one first and/or at least one second composite pigments are present in an amount ranging from 40 to 125 parts by weight, per 100 parts by weight of the core.

39. The set of at least two solid makeup compositions according to claim 1, wherein the at least one first and/or at least one second coloring substances of the at least one first and/or at least one second composite pigments comprises at least one organic pigment.

40. The set of at least two solid makeup compositions according to claim 1, wherein the at least one first and/or at least one second coloring substances of the at least one first and/or at least one second composite pigments is chosen from carmine cochineal; organic pigments of azo, anthraquinone, indigo, xanthene, pyrene, quinoline, triphenylmethane and fluorane dyes, and organic lakes; insoluble sodiums; potassium, calcium, barium, aluminum, zirconium, strontium and titanium salts; and acid dyes.

41. The set of at least two solid makeup compositions according to claim 40, wherein the at least one acid dye is chosen from azo, anthraquinone, indigo, xanthene, pyrene, quinoline, triphenylmethane and fluorane dyes, and other dyes comprising at least one carboxylic or sulfonic acid group.

42. The set of at least two solid makeup compositions according to claim 40, wherein the at least one first and/or at least one second coloring substances of the at least one first and/or at least one second composite pigments are organic pigments chosen from D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, and FD&C Yellow No. 6.

43. The set of at least two solid makeup compositions according to claim 40, wherein the at least one first and/or at least one second coloring substances of the at least one first and/or at least one second composite pigments are organic lakes chosen from D&C Red No. 2 Aluminum lake, D&C Red No. 3 Aluminum lake, D&C Red No. 4 Aluminum lake, D&C Red No. 6 Aluminum lake, D&C Red No. 6 Barium lake, D&C Red No. 6 Barium/Strontium lake, D&C Red No. 6 Strontium lake, D&C Red No. 6 Potassium lake, D&C Red No. 7 Aluminum lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminum lake, D&C Red No. 9 Barium lake, D&C Red No. 9 Barium/Strontium lake, D&C Red No. 9 Zirconium lake, D&C Red No. 10 Sodium lake, D&C Red No. 19 Aluminum lake, D&C Red No. 19 Barium lake, D&C Red No. 19 Zirconium lake, D&C Red No. 21 Aluminum lake, D&C Red No. 21 Zirconium lake, D&C Red No. 22 Aluminum lake, D&C Red No. 27 Aluminum lake, D&C Red No. 27 Aluminum/Titanium/Zirconium lake, D&C Red No. 27 Barium lake, D&C Red No. 27 Calcium lake, D&C Red No. 27 Zirconium lake, D&C Red No. 28 Aluminum lake, D&C Red No. 30 lake, D&C Red No. 31 Calcium lake, D&C Red No. 33 Aluminum lake, D&C Red No. 34 Calcium lake, D&C Red No. 36 lake, D&C Red No. 40 Aluminum lake, D&C Blue No. 1 Aluminum lake, D&C Green No. 3 Aluminum lake, D&C Orange No. 4 Aluminum lake, D&C Orange No. 5 Aluminum lake, D&C Orange No. 5 Zirconium lake, D&C Orange No. 10 Aluminum lake, D&C Orange No. 17 Barium lake, D&C Yellow No. 5 Aluminum lake, D&C Yellow No. 5 Zirconium lake, D&C Yellow No. 6 Aluminum lake, D&C Yellow No. 7 Zirconium lake, D&C Yellow No. 10 Aluminum lake, FD&C Blue No. 1 Aluminum lake, FD&C Red No. 4 Aluminum lake, FD&C Red No. 40 Aluminum lake, FD&C Yellow No. 5 Aluminum lake, and FD&C Yellow No. 6 Aluminum lake.

44. The set of at least two solid makeup compositions according to claim 40, wherein the at least one first and/or at least one second coloring substances of the at least one first and/or at least one second composite pigments comprises an organic lake supported by an organic support that comprises at least colophane and/or aluminum benzoate.

45. The set of at least two solid makeup compositions according to claim 1, wherein the at least one first and/or at least one second composite pigments comprise at least one binder.

46. The set of at least two solid makeup compositions according to claim 45, wherein the at least one binder comprises at least one compound chosen from silicone compounds, polymeric compounds, and oligomeric compounds comprising at least one organosilane, organosilanes of a fluoroalkylated organosilane, polysiloxanes, and coupling agents.

47. The set of at least two solid makeup compositions according to claim 46, wherein the at least one coupling agent is chosen from silanes, titanates, aluminates, and/or a zirconates.

48. The set of at least two solid makeup compositions according to claim 46, wherein the at least one binder comprises at least one silicone compound.

49. The set of at least two solid makeup compositions according to claim 1, wherein the cores of the at least one first and/or at least one second composite pigments are colored.

50. The set of at least two solid makeup compositions according to claim 1, wherein the at least one first and/or at least one second compositions do not comprise un-covered particles of titanium dioxide.

51. The set of at least two solid makeup compositions according to claim 1, wherein the at least one first and/or at least one second compositions further comprise at least one cosmetically and/or dermatologically active agent.

52. The set of at least two solid makeup compositions according to claim 1, wherein the at least one first and/or at least one second compositions further comprise at least one oil or at least one wax.

53. The set of at least two solid makeup compositions according to claim 17, wherein the at least one additional coloring substance comprises at least one coloring substance chosen from inorganic pigments, organic pigments, pearlescent pigments, liposoluble coloring agents and hydrosoluble coloring agents.

54. The set of at least two solid makeup compositions according to claim 53, wherein the at least one additional coloring substance comprises at least one inorganic pigment chosen from titanium dioxide, zirconium oxide, cerium oxide, iron oxide, chromium oxide, manganese violet, ultramarine blue, chromium hydrate, and iron blue.

55. The set of at least two solid makeup compositions according to claim 53, wherein the at least one additional coloring substance comprises at least one organic pigment chosen from carbon black, D&C type pigments, and lakes based on cochineal carmine, barium, strontium, calcium, and/or aluminum.

56. The set of at least two solid makeup compositions according to claim 53, wherein the at least one additional coloring substance comprises at least one white and/or colored pearlescent pigment.

57. The set of at least two solid makeup compositions according to claim 54, wherein the at least one additional coloring substance comprises at least one liposoluble colorant chosen from Sudan red, D&C Red No. 17, D&C Green No. 6, β-carotene, jojoba oil, Sudan brown, D&C Yellow No. 11, D&C Violet No. 2, D&C Orange No. 5, and quinoline yellow.

58. The set of at least two solid make up compositions according to claim 1, wherein the at least one first and at least one second coloring substances are not melanin.

59. The set of at least two solid make up compositions according to claim 1, wherein the at least one first and at least one second composite pigments each have a saturation C* greater than or equal to 30.

60. The set of at least two solid make up compositions according to claim 1, wherein the at least one first and at least one second composite pigments are not interference pigments.

61. The set of at least two solid makeup compositions according to claim 1, wherein the compositions are foundations.

62. The set of at least two solid makeup compositions according to claim 1, wherein the compositions are eye shadows.

63. The set of at least two solid makeup compositions according to claim 1, wherein the compositions are blushers.

64. The set of at least two solid makeup compositions according to claim 1, wherein the compositions are substances for concealing dark circles and/or bags under the eyes.

65. The set of at least two solid makeup compositions according to claim 1, wherein the compositions are body makeups.

66. The set of at least two solid makeup compositions according to claim 1, wherein the compositions are lipsticks.

67. The set of at least two solid makeup compositions according to claim 45, wherein the at least one binder is organic.

68. The set of at least two solid makeup compositions according to claim 45, wherein the at least one binder is present in an amount less than or equal to 5% by weight, relative to the total weight of the composite pigment.
